(19) United States
(12) Patent Application Publication
(10) Pub. No.: US 2016/0120713 A1
(43) Pub. Date: May 5, 2016

(54) PROTECTIVE COVERING FOR A PATIENT

(71) Applicant: David Scott Magbee, Murrieta, CA (US)

(72) Inventor: David Scott Magbee, Murrieta, CA (US)

(21) Appl. No.: 14/930,594

(22) Filed: Nov. 2, 2015

Related U.S. Application Data
(60) Provisional application No. 62/074,471, filed on Nov. 3, 2014.

(51) Int. Cl. A61G 1/04; A61F 5/37

(52) U.S. Cl. CPC A61G 1/04; A61F 5/37

(57) ABSTRACT

A protective sheet with a transparent region and a protective region formed from different materials. The protective sheet has an elasticated perimeter configured to secure the protective sheet onto a support platform for a human body. The protective sheet can be oriented so that the transparent region covers at least a head of the human body and the protective region covers at least portion of the human body below the head.

### CROSS-REFERENCE TO RELATED APPLICATIONS

The current application claims priority to U.S. Provisional Patent Application No. 62/074,471 filed on Nov. 3, 2014, which is incorporated herein by reference in its entirety.

### TECHNICAL FIELD

The subject matter described herein relates to a protective sheet for covering a human body.

### BACKGROUND

Protective sheets have been used in medical and emergency situations. The protective sheets are typically thin and flat, and made of paper or plastic sheets. They are typically difficult to keep secure to gurneys or spinal immobilization boards to keep the patient covered in unpleasant weather conditions, allowing the patient to be exposed to the elements.

### SUMMARY

In one aspect, a protective sheet is provided. The protective sheet can include a transparent region. The transparent region can be formed of a first material that is transparent. The transparent region can extend from a first end of the protective sheet. The protective sheet can include a protective region. The protective region can be formed of a second material that is different from the first material. The protective region can extend from a second end opposite the first end of the protective sheet and to the transparent region. The first material can contact the second material. The protective sheet can include an elasticated perimeter. The elasticated perimeter can be configured to secure the protective sheet onto a support platform for a human body. The protective sheet can be configured to be oriented so that the transparent region covers at least a head of the human body and the protective region covers at least portion of the human body below the head.

The protective sheet can optionally include one or more of the following features. The protective material of the protective region of the protective sheet can comprise an outer layer and an inner layer. The outer layer can be formed from a polyurethane laminate material. The inner layer of the protective material of the protective region can be formed from a tissue material.

The protective sheet may include an elasticated strip disposed adjacent to the first end of the protective sheet. The elastic strip may be disposed substantially parallel to the first end of the protective sheet. As used herein, parallel can mean actually parallel, or substantially parallel. The elasticated strip can be configured to cause the sides of the protective sheet to exert pressure on the support platform to retain the plastic sheet on the support platform.

The transparent material can include ventilation apertures configured to facilitate the ingress and egress of air to and from the inside of the protective sheet. The ventilation apertures can include a series of holes on one or more sides of the transparent material.

The protective sheet can include at least one curved rib. The at least one curved rib can be disposed laterally across the protective sheet. The at least one curved rib can be configured to cause the middle of the protective sheet to rise above the support platform to form a tunnel. The at least one curved rib can be formed of plastic.

The protective material can include at least one channel disposed transversely across the protective material. The at least one channel can be configured to support the at least one curved rib. The at least one channel can have closed ends or open ends.

The corners of the protective sheet can be rounded. The corners of the protective sheet can be configured to secure to the corners of the support platform.

In another aspect, a method of making a protective sheet is provided. The method can include providing a transparent region formed of a first material. The first material can be transparent and extend from a first end of the protective sheet. The method can include providing a protective region. The protective region can be formed of a second material that is different from the first material. The protective region can extend from a second end opposite the first end of the protective sheet and to the transparent region such that the first material contacts the second material. The method can include providing an elasticated perimeter configured to secure the protective sheet onto a support platform for a human body. The protective sheet can be configured to be oriented so that the transparent region covers at least a head of the human body and the protective region covers at least portion of the human body below the head.

The method can optionally include one or more of the following features. The protective region can comprise an inner layer and an outer layer. The transparent region can comprise ventilation apertures for facilitating ingress and egress of air.

The method can further comprise providing at least one curved rib disposed laterally across a width of the protective sheet. The at least one curved rib can be configured to cause the protective sheet to form a tunnel shape over a patient when the protective sheet is installed on a patient support platform. In some variations, the at least one curved rib can be formed of plastic tubing.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to an enterprise resource software system or other business software solution or architecture, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A is an illustration of a protective sheet having one or more elements consistent with the current description;

FIG. 1B is an illustration of a portion of a protective sheet having one or more elements consistent with the current description;

FIG. 1C is an illustration of a protective sheet having one or more elements consistent with the current description;

FIG. 2 is an illustration of a protective sheet being utilized on a gurney, the protective sheet having one or more elements consistent with the current description;

FIGS. 3A-3D are illustrations of portions of a protective sheet having one or more elements consistent with the current description;

FIG. 4 is a process flow diagram illustrating aspects of a method for making a protective sheet, the method having one or more element consistent with the current description; and,

FIG. 5 is an illustration of a protective bag having one or more elements consistent with the current description.

### DETAILED DESCRIPTION

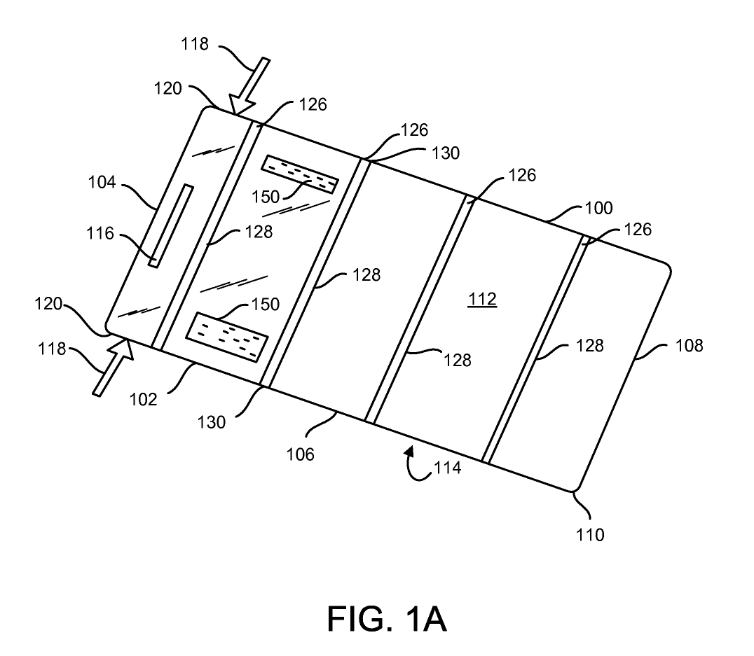

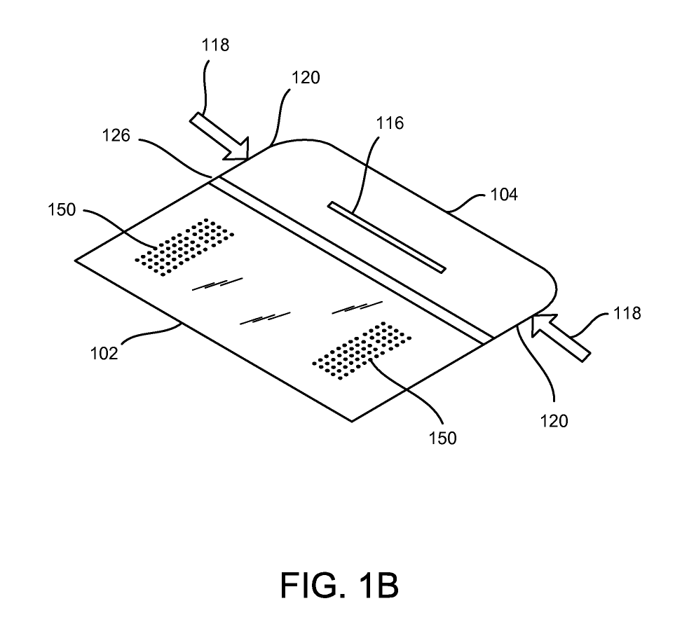

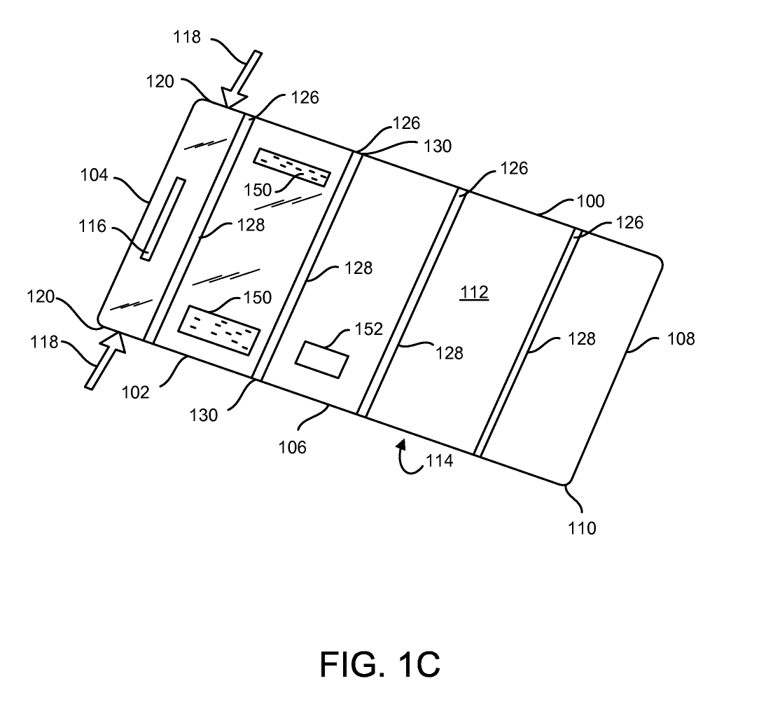

FIG. 1A is an illustration of a protective sheet 100 having one or more elements consistent with the current description. The protective sheet 100 can comprise a transparent region 102. FIG. 1B is an illustration of an example of a transparent region 102 of the protective sheet 100 having one or more elements consistent with the current description. The transparent region 102 can be formed of a first material. The first material can be transparent. The first material can extend from a first end 104 of the protective sheet. The first material can be a transparent polymer material, a transparent polyurethane material, a transparent polyethylene material, a transparent vinyl material, mylar, or the like.

The protective sheet 100 can include a protective region 106. The protective region '06 can be formed of a second material. The second material can be different from the first material. The second material can extend from a second end 108 opposite the first end 104 of the protective sheet 100. The second material can extend to the transparent region 102 such that the first material contacts the second material.

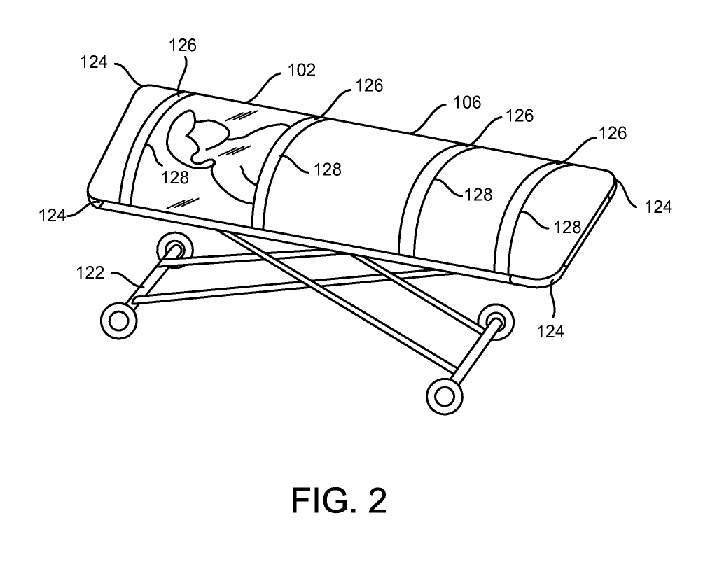

The protective sheet 100 can include an elasticated perimeter 110. The elasticated perimeter 110 can be configured to secure the protective sheet 100 onto a support platform for a human body (as shown in FIG. 2). For example, the elasticated perimeter 110 can be configured to secure the protective sheet 100 to the frame of a gurney, mattress of a gurney, a spinal board, a stretcher, or the like.

The protective sheet 100 can be configured to be oriented on the support platform so that the transparent region 102 covers at least a head of the human body and the protective region 106 covers at least portion of the human body below the head.

In some variations, the protective region 106 of the protective sheet 100 can comprise an outer layer 112 and an inner layer 114. The outer layer 112, for example, can be formed of polyurethane laminate material. The polyurethane laminate material can have one or more protective properties, including wind resistance, water resistance, warming properties, cooling properties, and the like. In some variations, the outer layer 112 can be formed from a composite material, a vinyl material, a polymer material, mylar, or the like.

The inner layer 114 of the protective region 106 can be formed, for example, of a tissue material. The tissue material can have one or more properties, including insulation, absorbency, softness, or the like.

In some variations, a material can be provided between the first end 104 of the protective sheet 100 and the transparent region 102. The material provided between the first end 104 of the protective sheet 100 and the transparent region 102 can be the same material as forms the protective region 106 of the protective sheet 100. In some variations, the material between the first end 104 and the transparent region 102 can be the same material as forming the transparent region 102.

The transparent region 102 can comprise an elasticated strip 116. The elasticated strip 116 can be disposed adjacent the first end 104 of the protective sheet 100. The elasticated strip can be configured to be disposed substantially parallel to the first end 104 of the protective sheet 100. The elasticated strip 116 can be configured to exert a force 118 onto the sides 120 of the transparent region 102. The force 118 exerted onto the sides 120 of the transparent region can cause the protective sheet to hug the support platform (as shown in FIG. 2).

FIG. 2 is an illustration of a protective sheet 100 being utilized on a gurney 122, the protective sheet 100 having one or more elements consistent with the current description. While a gurney is illustrated in FIG. 2, the presently described subject matter can be used on any human body support platform. For example, a hospital bed, a spinal board, an operating table, or the like. The corners 124 of the protective sheet 100 can be rounded. The rounded corners 124 can be configured to grip the corners of the human body support platform.

The protective sheet 100 can include one or more ribs 126. The one or more ribs 126 can be disposed transversely across the protective sheet 100. The one or more ribs 126 can be curved. The one or more ribs 126 can be configured to cause the central core of the protective sheet 100 to be raised above a patient when the protective sheet 100 is attached to the support platform. The one or more ribs 126 can be configured to cause the protective sheet 100 to form a tunnel on the support platform. The one or more ribs 126 can be configured to raise the protective sheet 100 above the patient when the protective sheet 100 is secured to the support platform. The one or more ribs 126 can include strips, pipes, tubes, or the like. The one or more ribs 126 can be formed from plastic. The one or more ribs 126 can be formed from tubing, tabs, ties, strips, or the like. In some variations, the one or more ribs 126 can be formed contiguously with the protective sheet 100. For example, the outer layer 112 can be formed from a material capable of being formed with different thicknesses and/or densities in different regions of the material.

In some variations, the protective sheet 100 can comprise at least one channel 128 disposed transversely across the protective sheet 100. The at least one channel 128 can be configured to contain a rib 126. The at least one channel 128 can be configured to cause the rib 126 to be maintained within the channel 128. In some variations, the at least one channel 128 can have closed ends 130.

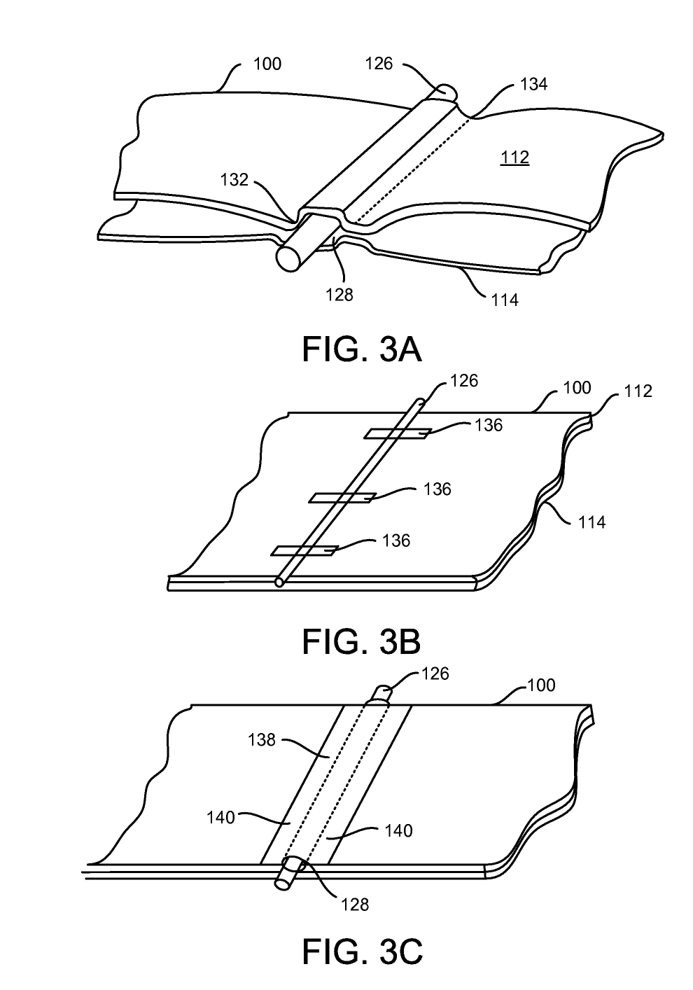

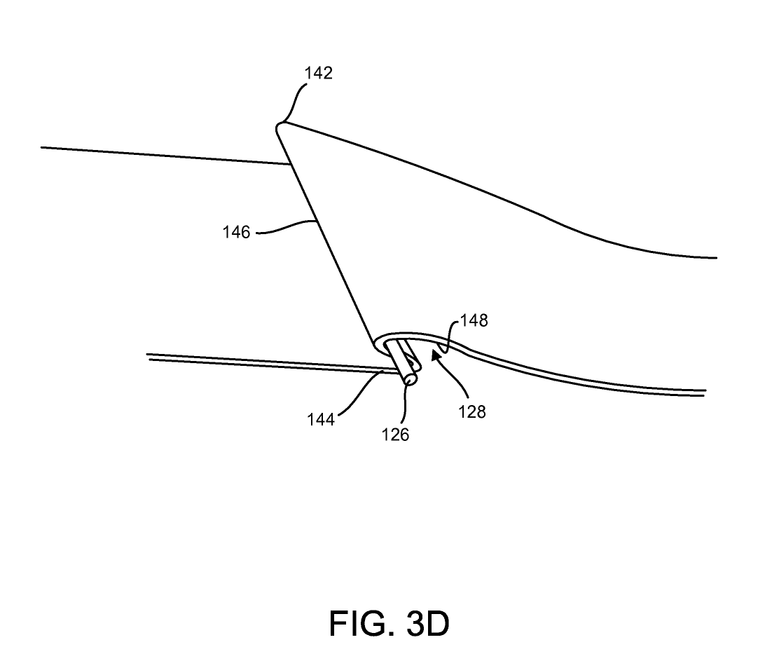

FIG. 3A is an illustration showing a portion of a protective sheet 100 having one or more features consistent with the current description. The at least one channel 128 can be formed by attaching the outer layerl 12 and the inner layer 114 transversely across the protective sheet 100. The outer layer 112 and the inner layer 114 can be attached along two substantially parallel tracks 132, 134 to cause the creation of a channel 128 between the two substantially parallel tracks 132, 134. The two substantially parallel tracks 132, 134 can be positioned to cause the channel 128 to be capable of housing the one or more ribs 126. The outer layer 112 and the inner layer 114 can be attached together by stitching, plastic welding, adhesive, by integrating the layers during the manufacturing process, or the like.

In some variations, the one or more ribs 126 can be attached to the protective sheet 100 with an adhesive. In some variations, the one or more ribs 126 can be attached to the protective sheet 100 by plastic welding the one or more ribs 126 to the outer layer 112 of the protective sheet 100

FIG. 3B is an illustration of a portion of a protective sheet 100 having one or more elements consistent with the current description. In some variations, the one or more ribs 126 can be positioned transversely across the protective sheet 100 and attached to the protective sheet 100 at attachment points 136 intermittently disposed along the length of the one or more ribs 126. The attachment points 136 intermittently disposed along the length of the one or more rib 126 can be stiches, plastic welds, material patches, or the like.

FIG. 3C is an illustration of a portion of a protective sheet 100 having one or more elements consistent with the current description. In some variations, the protective sheet 100 can include a strip of material 138. The strip of material 138 can be positioned laterally across the protective sheet 100. The strip of material 138 can be attached to the outer layer 112 of the protective sheet 100. The strip of material 138 can be attached to the outer layer 112 at the edges 140 of the strip of material 138. The strip of material 138 can be attached using an adhesive, stitching, plastic welding, or the like.

FIG. 3D is an illustration of a portion of a protective sheet 100 having one or more elements consistent with the current description. In some variations, a portion 142 of the protective sheet can be folded upon itself such that it overlaps a different portion 144 of the protective sheet. A leading edge 146 of the folded portion 142 can be attached to a corresponding portion 148 of the protective sheet 100. The leading edge 146 can be attached by stitching, plastic welding, using an adhesive, or the like. Folding a portion of the protective sheet 100 upon itself can form a channel 128 for receiving a rib 126. In some variations, one or more ribs 126 can be provided and positioned laterally across the protective sheet 100 during the manufacturing process. The protective sheet 100 can then be folded around the one or more ribs 126. The protective sheet 100 can have as many folds as there are ribs 126. In some variations, the one or more ribs 126 may be introduced into the channel(s) 128 of the protective sheet 100 after the folds have been formed.

In some variations, the one or more ribs 126 may have an adhesive applied to at least a portion of the one or more ribs 126. When the rib(s) 126 are inserted into the channel(s) 128, the adhesive on the rib(s) can adhere to the inside of the channel(s) 128 causing the rib(s) 126 to be retained in-place in the channel(s) 126.

While FIG. 3D shows the rib being disposed in a fold of the outer layer 112 of the protective sheet 100, such that the rib would be disposed on the outside of the protective sheet 100, the rib can be disposed in a fold, such that the rib would be disposed on the inside of the protective sheet 100.

When provided, the rib(s) 126 can be disposed in the transparent region 102, the protective region 106, or both. In some variations, the channel(s) 128 formed in the protective sheet 100 can be closed-ended. In some variations, the channel(s) 128 formed in the protective sheet 100 can be open-ended.

FIG. 1B is an illustration of the transparent region 102 of the protective sheet 100 having one or more elements consistent with the current description. The transparent region 102 can be formed from a transparent material to allow the patient to see out of the protective sheet 100. This allows the patient's head to be covered and protected from the elements while allowing the patient to see their surroundings. Being able to observe surroundings facilitates the patient in reducing their anxiety. The transparent region 102 can comprise one or more ventilation apertures 150. The one or more ventilation apertures 150 can comprise at least one hole configured to facilitate the ingress and egress of air and/or moisture. In some variations, the one or more ventilation apertures 150 can be a mesh, an aperture covered by a flap, and/or other apertures. The one or more ventilation apertures 150 can include two sets of ventilation apertures disposed on either side of the central axis of the transparent region 102.

The transparent region 102 of the protective sheet 100 can be configured to facilitate persons outside of the protective sheet 100 to visually monitor the patient. A patient's mental status, anxiety level, or general health status can change over time. For example, a patient may start to vomit, lose consciousness, or the like.

The transparent region 102 of the protective sheet 100 can be configured to facilitate verbal communication with the patient. The one or more ventilation apertures 150 can be configured to facilitate verbal communication between the patient and a medical provider.

FIG. 1C is an illustration of a protective sheet 100 having one or more therapy ports 152, the protective sheet having one or more. The therapy port 152 can be configured to facilitate the provision of one or more therapies. For example, the therapy port 152 can be configured to facilitate the entry of tubes. Tubes may be required for providing oxygen therapy to the patient. While the therapy port 152 is illustrated as being in the protective region 106 of the protective sheet 100, this is not intended to be limiting. A therapy port(s) 152 can be disposed in the transparent region 102, protective region 106, or both. Multiple therapy ports 152 can be provided. The therapy port(s) 152 can be configured to facilitate providing therapies while minimizing the patient's exposure to outside elements. The therapy port(s) 152 can be formed from multiple overlapping layers.

The therapy port(s) 152 can be configured to facilitate providing temperature regulating gasses to the patient within the protective sheet 100. When in cold environments, warm air can be passed into the protective sheet 100. When in hot environments, cool air can be passed into the protective sheet 100.

In some variations, equipment for providing therapy to a patient can be passed between the perimeter and the human support platform. A medical provider can lift up a portion of the perimeter of the protective sheet 100 and place the therapy equipment therebetween.

While the presently described protective sheet 100 is intended to fit any size and shape of human support platform, such as a gurney, hospital bed, spinal board, and the like, the following provides a non-limiting exemplary implementation of the presently described subject matter.

In some variations, the transparent region 102 may have a width between 40 and 48 inches in width. For some applications, the transparent region 102 may have a width of approximately 44 inches. The transparent region 102 may have a length of between 32 and 40 inches, between 30 and 38 inches, or the like. In some applications, the transparent region 102 may have a length of 36 inches, 34 inches, or the like.

In some variations, the protective region 106 of the protective sheet 100 may have a width of between 40 and 48 inches. In some applications the protective region may have a width of about 44 inches. The protective region 106 may have a length of between 72 and 78 inches, 69 and 75 inches, or the like. In some applications, the protective region 106 may have a length of about 74 inches, 71 inches, or the like. In some exemplary applications, the protective sheet may have a length of about 110 inches, 105 inches, 100 inches, or the like.

In some variations, the transparent region 102 and the protective region 106 can be attached together and include a channel 128 for receiving a rib 126. In some applications, the rib can be a flexible tube or tie. In some applications, the flexible tube or tie can be ¼ inch in diameter.

In some variations, the elastic perimeter 110 can be provided around the entire perimeter. In some variations, the elastic perimeter 110 can be provided in the vicinity of the corners of the protective sheet 100. In an exemplary variation, the elastic perimeter 110 can include an elastic stitch or elastic band. The elastic stitch or band can be sewn into the perimeter of the protective sheet 100. In some variations, the corners of the protective sheet 100 can be rounded off by between 2 inches and 10 inches. In an exemplary application the corners can be rounded off at approximately 6 inches. When provided, the elasticated strip 116 can be between 4 and 12 inches from the first end 102. In some applications, the elasticated strip 116 can be approximately 8 inches from the first end 102.

In some applications, the protective sheet 100 can include channels 128 configured to receive or house ribs 126. The ribs can be about ¼ inch diameter flexible tubes or ties. In some applications the ribs 126 can be between 34 and 42 inches in length. In an exemplary application the ribs can be about 38 inches in length. In one embodiment five channels may be provided disposed along the length of the protective sheet. The transparent region may include at least one channel. The protective region may include at least three channels. The intersection of the transparent region and the protective region may include at least one channel. One or more ribs may be disposed in the channels.

The protective sheet 100 can be configured to be collapsed along a longitudinal axis of the protective sheet 100 for storage. Ribs disposed in the protective sheet can be resilient, such that the ribs can be bent or folded for storage, and return to their original shape when the protective sheet is deployed.

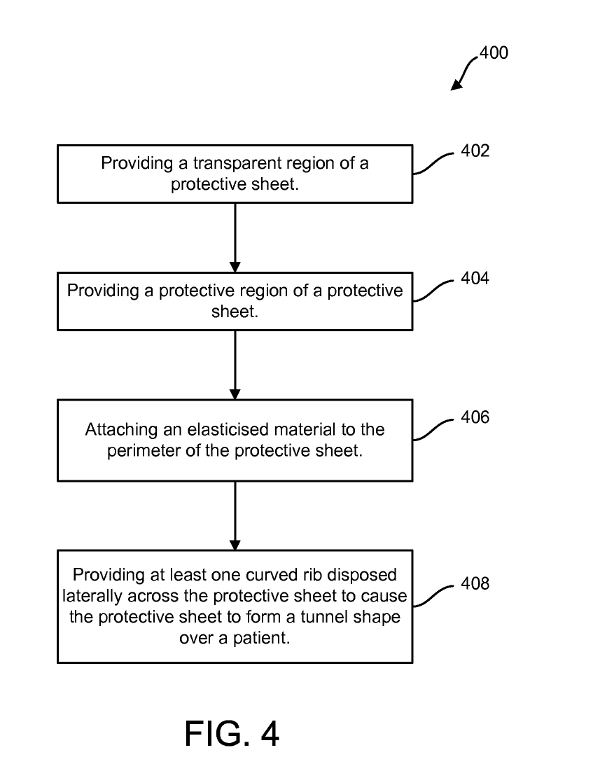

FIG. 4 is a process flow diagram illustrating aspects of a method 400 for making a protective sheet, the method having one or more element consistent with the current description.

At 402, a transparent region of a protective sheet, such as protective sheet 100, can be formed. The transparent region can be formed form first material. The first material can be transparent. The transparent region can extend from a first end of the protective sheet 100.

At 404, a protective region of a protective sheet, such as protective sheet 100, can be formed. The protective region can be formed from a second material. The second material can be different from the first material. The protective region can extend from a second end opposite the first end of the protective sheet. The protective region can extend to the transparent region, such that the first material contacts the second material.

At 406, an elasticated material can be attached to the protective sheet. The elasticated material can be attached to the perimeter of the protective sheet. The elasticated material can be configured to secure the protective sheet onto a support platform for a human body.

In some variations, the protective sheet can be configured to be oriented so that the transparent region covers at least a head of the human body and the protective region covers at least portion of the human body below the head.

At 408, at least one curved rib can be disposed laterally across the protective sheet. The at least one curved rib can be disposed on the transparent region, the protective region, or in both regions of the protective sheet. The at least one curved rib can be configured to cause the middle of the protective sheet to rise above the support platform to form a tunnel.

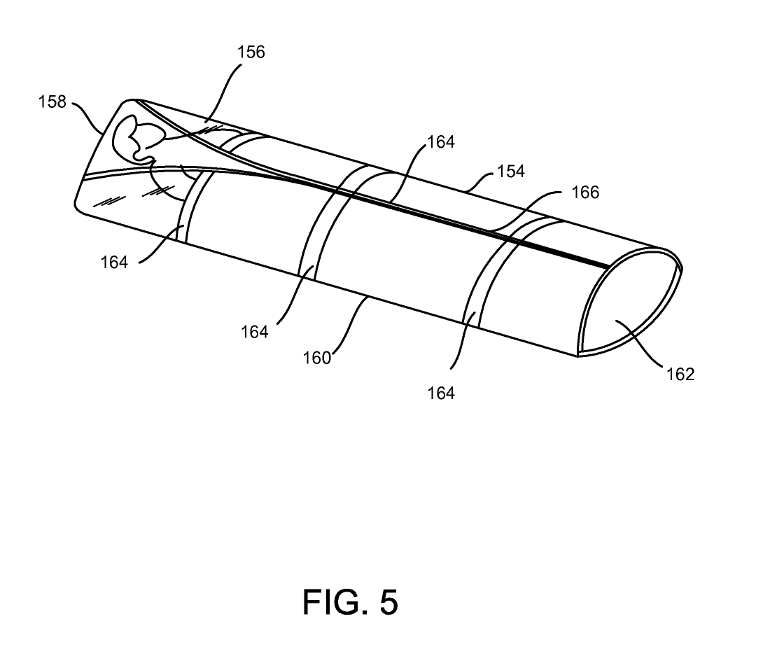

FIG. 5 is an illustration of a protective bag 154 for containing a patient having one or more elements consistent with the current description. The protective bag 154 can comprise a transparent region 156 extending from a first end 158. The transparent region 156 can be formed of a first material. The first material can be transparent. The first material can extend from a first end 158 of the protective bag 154.

The protective bag 154 can include a protective region 160. The protective region 160 can be formed of a second material. The second material can be different from the first material. The second material can extend from a second end 162 opposite the first end 158 of the protective bag 154. The second material can extend to the transparent region 156 such that the first material contacts the second material.

The protective bag 154 can include an closing device 164. The closing device 164 can form a seal when closed to protect the patient inside the protective bag 154 from the elements.

The protective bag 154 can be configured to be oriented so that the transparent region 156 covers at least a head of the human body and the protective region 160 covers at least portion of the human body below the head.

In some variations, the protective region 160 of the protective bag 154 can comprise an outer layer and an inner layer, similar to the protective sheet 100 of FIG. 1A. The protective region 160 can be formed from, for example, a polyurethane laminate material. The polyurethane laminate can have one or more protective properties, including wind resistance, water resistance, warming properties, cooling properties, or the like.

The inner layer of the protective region 160 can be formed, for example, of a tissue material. The tissue material can have one or more properties, including insulation, absorbency, softness, or the like.

The protective bag 154 can include one or more ribs 164. The one or more ribs 164 can be disposed in one or more channels, similarly to the one or more ribs of the protective sheet 100, shown in FIG. 1A. The one or more ribs 164 can be configured to cause the middle portion 166 of the protective bag 154 to be raised above the patient. The protective bag 154 can be configured to quarantine the patient in the protective bag from other people. The protective bag 154 can be configured to be used in hazardous situations to increase safety of the patient and those surrounding the patient.

The presently described subject matter provides one or more non-limiting benefits. For example, the presently described protective sheet can be configured to facilitate keeping the elements from the patient. The presently describe protective sheet can be configured to keep the elements from all of the patient's body parts, including the head and upper torso while allowing medical workers and emergency responders to monitor the patient and provide therapy to the patient. The presently described subject matter includes a protective sheet configured keep a patient from being subjected to wind, rain, snow, and the like.

Although a few variations have been described in detail above, other modifications are possible. For example, the logic flow depicted in the accompanying figures and described herein do not require the particular order shown, or sequential order, to achieve desirable results. In addition, other processing attributes other than threads can be used to determine whether to selectively change the speed of a core scheduled to process the corresponding tasks. Moreover, the term task can be construed, unless explicitly stated otherwise, to include jobs and other broader groupings of related computing activities. Other embodiments may be within the scope of the following claims.

What is claimed is:

1. A protective sheet comprising:
a transparent region formed of a first material that is transparent and extending from a first end of the protective sheet;
a protective region formed of a second material that is different from the first material and extending from a second end opposite the first end of the protective sheet and to the transparent region such that the first material contacts the second material; and,
an elasticated perimeter configured to secure the protective sheet onto a support platform for a human body; and,
wherein the protective sheet is configured to be oriented so that the transparent region covers at least a head of the human body and the protective region covers at least portion of the human body below the head.

2. The protective sheet of claim 1, wherein the protective material comprises an outer layer and an inner layer.

3. The protective sheet of claim 2, wherein the outer layer is a polyurethane laminate material.

4. The protective sheet of claim 2, wherein the inner layer is a tissue material.

5. The protective sheet of claim 1, further comprising:
an elasticated strip disposed adjacent and parallel to the first end of the protective sheet, the elasticated strip configured to cause the sides of the protective sheet to exert pressure on to the support platform to retain the plastic sheet on the support platform.

6. The protective sheet of claim 1, wherein the transparent material comprises ventilation apertures configured to facilitate the ingress and egress of air to and from the inside of the protective sheet.

7. The protective sheet of claim 1, wherein the ventilation apertures comprise of a series of holes on one or more sides of the transparent material.

8. The protective sheet of claim 1, further comprising:
at least one curved rib disposed laterally across the protective sheet, the at least one curved rib configured to cause the middle of the protective sheet to rise above the support platform to form a tunnel.

9. The protective sheet of claim 8, wherein the at least one curved rib is formed of plastic.

10. The protective sheet of claim 8, wherein the protective material comprises at least one channel disposed transversely across the protective material, the at least one channel configured to support the at least one curved rib.

11. The protective sheet of claim 10, wherein the at least one channel has closed ends.

12. The protective sheet of claim 1, wherein the corners of the protective sheet are rounded and configured to secure to the corners of the support platform.

13. A method of making a protective sheet, the method comprising:
providing a transparent region formed of a first material that is transparent and extending from a first end of the protective sheet;
providing a protective region formed of a second material that is different from the first material and extending from a second end opposite the first end of the protective sheet and to the transparent region such that the first material contacts the second material; and,
providing an elasticated perimeter configured to secure the protective sheet onto a support platform for a human body, wherein the protective sheet is configured to be oriented so that the transparent region covers at least a head of the human body and the protective region covers at least portion of the human body below the head.

14. The method of claim 13, wherein the protective region comprises an inner layer and an outer layer.

15. The method of claim 13, wherein the transparent region comprises ventilation apertures for facilitating ingress and egress of air.

16. The method of claim 13, further comprising:
providing at least one curved rib disposed laterally across a width of the protective sheet, the at least one curved rib configured to cause the protective sheet to form a tunnel shape over a patient when the protective sheet is installed on a patient support platform.

17. The protective sheet of claim 16, wherein the at least one curved rib is formed of plastic tubing.
